(19) United States
(12) Patent Application Publication
(10) Pub. No.: US 2025/0295318 A1
(43) Pub. Date: Sep. 25, 2025

(54) DETECTION APPARATUS

(71) Applicant: PARAMOUNT BED CO., LTD., Tokyo (JP)

(72) Inventors: Saori Toyota, Tokyo (JP); Takamasa KOGURE, Tokyo (JP); Takatoshi KASAI, Tokyo (JP); Ryo NAITO, Tokyo (JP); JUN SHITARA, Tokyo (JP)

(21) Appl. No.: 18/750,261

(22) Filed: Jun. 21, 2024

(30) Foreign Application Priority Data
Mar. 25, 2024 (JP) 2024-047986

(51) Int. Cl. A61B 5/024; A61B 5/0205; A61B 5/0245

(52) U.S. Cl. CPC A61B 5/02405; A61B 5/0205; A61B 5/0245

(57) ABSTRACT

A detection apparatus includes a biosignal acquisition unit configured to acquire a biosignal of a user, and a controller. The controller is configured to calculate one or more parameter values related to breathing of the user based on the biosignal acquired from the biosignal acquisition unit, and detect a presence of a symptom of aggravation of cardiac failure in the user when a calculated parameter value in which a cumulative value of previous day difference data has exceeded a predetermined threshold is present, among the calculated parameter values.

### BACKGROUND OF THE INVENTION

### Field

The present disclosure relates to a detection apparatus and the like.

### Background

The inventions in which whether an abnormality occurs in a user is determined from a biological information value and the like on the user have been known.

### BRIEF DESCRIPTION OF THE DRAWINGS

FIG. 1 is a diagram illustrating the whole in a first embodiment.

FIG. 2 a diagram illustrating a functional configuration of a hardware in the first embodiment.

FIG. 3A is a diagram illustrating a functional configuration of software in the first embodiment. FIG. 3B is a diagram illustrating one example of a threshold table in the first embodiment.

FIG. 4 is a diagram illustrating one example of parameter values in the first embodiment.

FIG. 5 is an operation flow illustrating processing in the first embodiment.

FIG. 6 is an operation flow illustrating processing in the first embodiment.

FIG. 7 is an operation flow illustrating processing in the first embodiment.

FIG. 8 is a diagram illustrating one example of parameter values in the first embodiment.

FIGS. 9A-9C are diagrams illustrating an example in the first embodiment.

FIGS. 10A-10C are diagrams illustrating an example in the first embodiment.

FIG. 11 is an operation flow illustrating processing in a second embodiment.

FIGS. 12A-12C are diagrams illustrating an example in the second embodiment.

FIGS. 13A-13C are diagrams illustrating an example in the second embodiment.

FIGS. 14A and 14B are diagrams illustrating an example in the second embodiment.

FIG. 15 is a diagram illustrating an example in a third embodiment.

FIGS. 16A and 16B are diagrams illustrating an example in the third embodiment.

### DESCRIPTION OF EMBODIMENTS

One or more embodiments are now described with reference to the drawings, wherein like reference numerals are used to refer to like elements throughout. In the following description, for purposes of explanation, numerous specific details are set forth in order to provide a thorough understanding of the various embodiments. It is evident, however, that the various embodiments can be practiced without these specific details (and without applying to any particular networked environment or standard).

As used in this disclosure, in some embodiments, the terms “component”, “system” and the like are intended to refer to, or comprise, a computer-related entity or an entity related to an operational apparatus with one or more specific functionalities, wherein the entity can be either hardware, or a combination of hardware and software in execution.

One or more components may reside within a process and/or thread of execution and a component may be localized on one computer and/or distributed between two or more computers. In addition, these components can execute from various computer readable media having various data structures stored thereon. The components may communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software application or firmware application executed by a processor, wherein the processor can be internal or external to the apparatus and executes at least a part of the software or firmware application. As yet another example, a component can be an apparatus that provides specific functionality through electronic components without mechanical parts, the electronic components can comprise a processor therein to execute software stored on a non-transitory electronic memory or firmware that confers at least in part the functionality of the electronic components. While various components have been illustrated as separate components, it will be appreciated that multiple components can be implemented as a single component, or a single component can be implemented as multiple components, without departing from example embodiments. Further, the various embodiments can be implemented as a method, apparatus or article of manufacture using standard programming and/or engineering techniques to produce software, firmware, hardware or any combination thereof to control a computer to implement the disclosed subject matter. The term “article of manufacture” as used herein is intended to encompass a computer-readable (or machine-readable) device or computer-readable (or machine-readable) storage/communications media having a computer program stored thereon. For example, computer readable storage media can comprise, but are not limited to, magnetic storage devices (e.g., hard disk, floppy disk, magnetic strips), optical disks (e.g., compact disk (CD), digital versatile disk (DVD)), smart cards, and flash memory devices (e.g., card, stick, key drive). Of course, those skilled in the art will recognize many modifications can be made to this configuration without departing from the scope or spirit of the various embodiments.

In addition, the words “example” and “exemplary” are used herein to mean serving as an instance or illustration. Any embodiment or design described herein as “example” or “exemplary” is not necessarily to be construed as preferred or advantageous over other embodiments or designs. Rather, use of the word example or exemplary is intended to present concepts in a concrete fashion. As used in this application, the term “or” is intended to mean an inclusive “or” rather than an exclusive “or”. That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Embodiments described herein can be exploited in substantially any wireless communication technology, comprising, but not limited to, wireless fidelity (Wi-Fi), global system for mobile communications (GSM), universal mobile telecommunications system (UMTS), worldwide interoperability for microwave access (WiMAX), enhanced general packet radio service (enhanced GPRS), third generation partnership project (3GPP) long term evolution (LTE), third generation partnership project 2 (3GPP2) ultra mobile broadband (UMB), high speed packet access (HSPA), Z-Wave, Zigbee and other 802.XX wireless technologies and/or legacy telecommunication technologies.

In general, one aspect of the present application is a detection apparatus including a biosignal acquisition unit configured to acquire a biosignal of a user, and a controller. The controller is configured to calculate one or more parameter values related to breathing of the user based on the biosignal acquired from the biosignal acquisition unit, and detect a presence of a symptom of aggravation of cardiac failure in the user when a calculated parameter value in which a cumulative value of previous day difference data has exceeded a predetermined threshold is present, among the calculated parameter values.

A description will hereinafter be made on one embodiment for implementing the present disclosure with reference to the drawings. Specifically, a case where an abnormality determination apparatus according to the present disclosure is applied will be described, however, the range to which the present disclosure is applied is not limited to the embodiment.

Generally, systems that use sleep measuring devices to predict changes in physical conditions are known. Such systems can detect not only cardiac failure but also changes in the physical conditions of patients.

In recent years, in Japan where an increase in cardiac failure patients caused by super-aging and cardiac failure pandemic are expected, it is expected that non-specialists make medical examinations as primary care doctors for the cardiac failure patients. In a situation where the number of support persons per one patient decreases due to the labor shortage in the whole society, support systems that watch patients and detect aggravation symptoms are demanded.

Although the cardiac failure patient describes subjective symptoms, and describes daily measurement results of a blood pressure and a body weight into a cardiac failure notebook to self-check own symptoms, a cardiac failure aggravation symptom has been difficult to be found due to the variations of the subjective impressions and the extent of the daily entry and management abilities of the patient himself/herself, and the shortage of knowledge and experiences of the non-medical specialist.

In order to solve such problems, a system and an apparatus that appropriately detect and make a notice of a symptom of cardiac failure aggravation of a user who is a patient will be described using embodiments below.

### 1. First Embodiment

### 1.1 Entire System

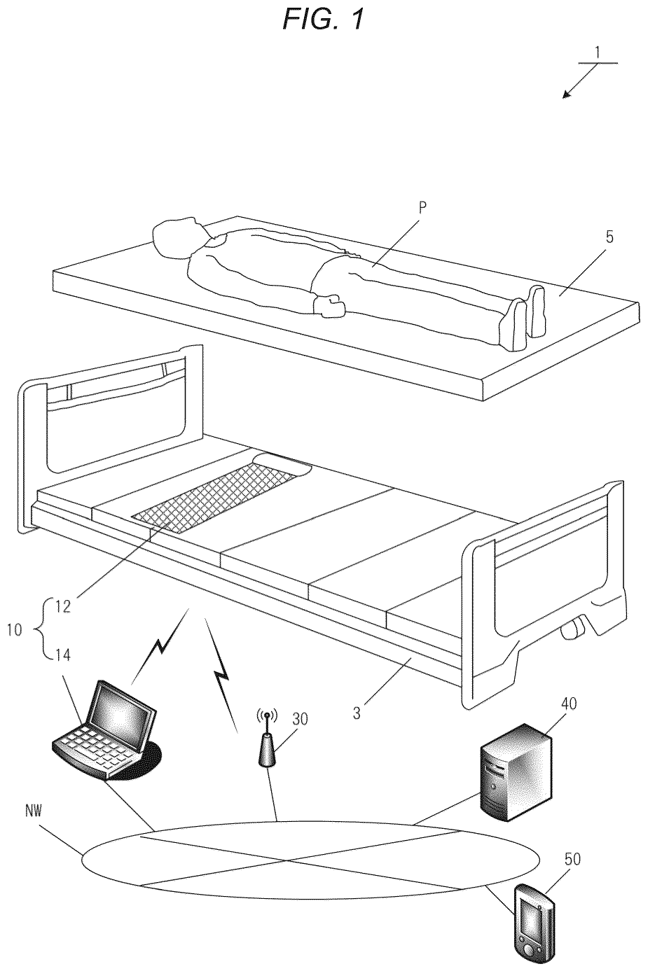

FIG. 1 is a diagram illustrating the whole overview of a detection system 1 to which an abnormality determination apparatus according to the present disclosure is applied. As illustrated in FIG. 1, the detection system 1 includes, for example, a detection apparatus 10 that detects a symptom of cardiac failure aggravation of a user P who is a patient. The detection apparatus 10 may be provided with a detection device 12 that is placed between sections of a bed 3 and a mattress 5, and a processor 14 for processing a value output from the detection device 12. The detection apparatus 10 may include, for example, the detection device 12 standalone having the function of the processor 14.

When a user (hereinafter, referred to as “user P” as one example) is present on the mattress 5, the detection device 12 detects a body vibration (vibration emitted from a human body) as a biosignal from the user P who is a patient. A biological information value of the user P is then calculated based on the detected vibration. In the present embodiment, the detection apparatus 10 may output and display the calculated biological information values (at least, a respiratory rate, a heartbeat rate, and an amount of activity), as biological information values of the user P. The processor 14 may include a general device, and thus is not limited to an information processor such as a computer, but may include a device, for example, a tablet or a smartphone.

The user is a person who has a disease of cardiac failure, and may be a person under medical treatment of the disease or a person who needs care. The user may be a person who needs no care, or may be an elderly person or a child.

The detection device 12 herein is formed in a sheet shape so as to have a thin thickness. This allows the detection device 12 even to be used without giving a discomfort feeling to the user P when the detection device 12 is placed between the bed 3 and the mattress 5, so that biological information values in the bed portion can be measured for long periods. In other words, biological information values and the like are acquired as a state of a user when the user is in bed-rest and at rest.

The detection device 12 only needs to acquire biosignals (a body movement, a respiratory motion, a ballistocardioaction, and the like) of the user P. In the present embodiment, the respiratory rate and the heartbeat rate are calculated based on the body vibration, but, for example, may be detected using an infrared sensor, a biosignal of the user P may be acquired from the acquired video and the like, or an actuator with a distortion gage may be used. The detection device 12 may be implemented as a smartphone, a tablet, or the like that is placed on the bed 3 (or the mattress 5), for example, by using the incorporated acceleration sensor or the like.

The bed 3 is installed in a variety of places. For example, the bed 3 is installed in a home of a user who is a patient, or is installed in a hospital in which the user is hospitalized or a facility in which the user resides.

The detection apparatus 10 is communicable with another device via a network NW. The detection device 12 in the detection apparatus 10 may be connected to the network NW, for example, via the processor 14, or the detection device 12 may be directly connected to the network NW via an access point 30 through a wireless LAN or the like. The detection device 12 may be directly connected to the network NW, for example, by a communication module communicable with movement communication networks (LTE/4G/5G/6G and the like) being incorporated therein.

The network NW is connectable with, for example, a server device 40 and a terminal device 50. The server device 40 may store, for example, a biological information value or the like acquired by the detection apparatus 10. The server device 40 may be, for example, an electronic medical record server that stores disease information on a user.

For example, the terminal device 50 may be an information processor, such as a smartphone, a tablet, and a laptop computer, that is used by a medical staff such as a medical doctor and a nurse. The terminal device 50 may be an information processor that is used by a staff in the facility, a family, and other persons. The terminal device 50 may be an information processor that is used by a patient himself/herself for a self-check.

### 1.2 Functional Configuration

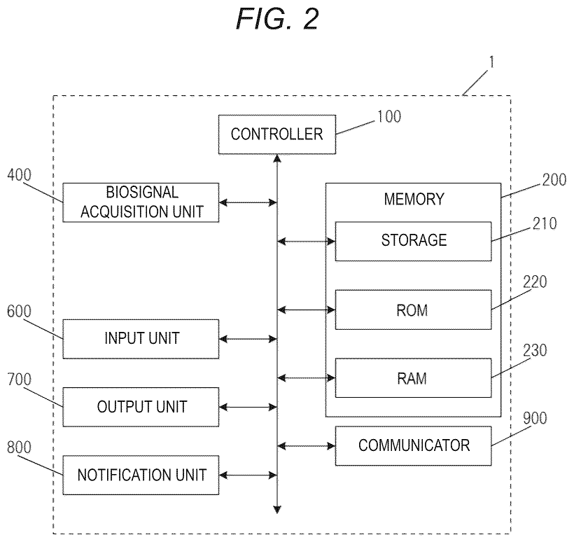

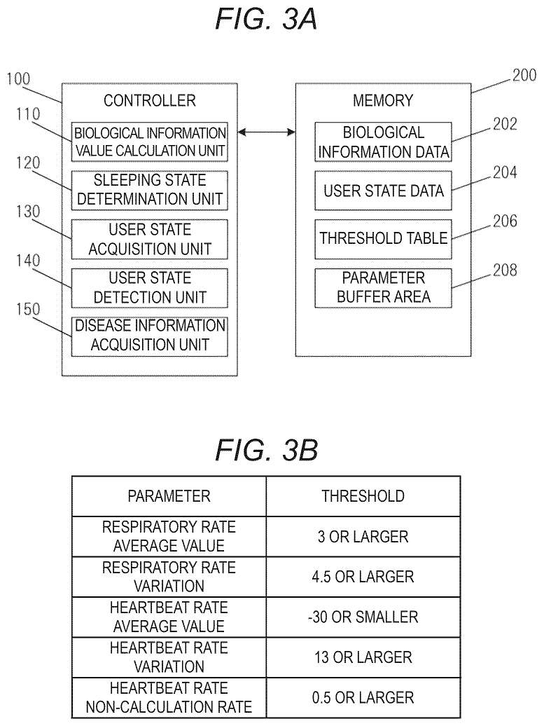

Next, a functional configuration of the detection apparatus 10 in the detection system 1 will be described using FIGS. 2 and 3. The detection apparatus 10 in the present embodiment includes the detection device 12 and the processor 14, and the respective functional units (processes) other than a biosignal acquisition unit 400 may be implemented by either of them. These devices are combined to function as the detection apparatus 10.

The detection apparatus 10 may perform a report (notification) operation after detecting a symptom of cardiac failure aggravation of a user. In this time, a report destination may be a staff, a patient himself/herself, or a family. As a reporting method, a report (notification) may be made simply by means of sounds or a screen display, or may be made to the terminal device by means of an email or the like. A report (notice) may be made to another terminal device or the like.

### 1.2.1 Hardware Configuration

As illustrated in FIG. 2, the detection apparatus 10 includes a controller 100, a memory 200 (a storage 210, a ROM 220, and a RAM 230), the biosignal acquisition unit 400, an input unit 600, an output unit 700, a notification unit 800, and a communicator 900, the number of each unit being one or plural as necessary.

In the case of FIG. 1, the detection device 12 is provided with the controller 100, the biosignal acquisition unit 400, and the memory 200, and the processor 14 may be provided with the other units.

The controller 100 controls the whole of the detection apparatus 10. The controller 100 implements various functions by reading and executing various programs stored in the memory 200 (for example, the storage 210 or the ROM 220) serving as a storage device. The controller 100 may be implemented by one or a plurality of controllers/calculation devices (a central processing unit (CPU) and a system on a chip (SoC)). The controller 100 may include a control circuit.

The memory 200 stores data on various information. The memory 200 is generally a device including one or more of the storages 210, the ROMs 220, and the RAMs 230, and stores data in any of which as necessary.

The storage 210 is a nonvolatile storage device that can store the programs and data. For example, the storage 210 may include a storage device such as a hard disk drive (HDD) and a solid state drive (SSD). The storage 210 may be an externally connectable USB memory or memory card. The storage 210 may be, for example, a storage area on a cloud.

The ROM 220 is nonvolatile memory capable of keeping the program and the data even when power is turned off.

The RAM 230 is a main memory that is mainly used by the controller 100 when executing the processing. The RAM 230 is a rewritable memory that temporarily keeps the programs read from the storage 210 and the ROM 220 and the data including results at the time of execution.

The biosignal acquisition unit 400 acquires a biosignal of the user P. In the present embodiment, as one example, a sensor that detects a change in pressure is used to acquire a body vibration that is one type of a biosignal. The controller 100 then converts the acquired body vibration into biological information value data such as a respiratory rate, a heartbeat rate, and an amount of activity, and outputs the converted biological information value data. In addition, based on the body vibration data acquired by the biosignal acquisition unit 400, the controller 100 can acquire a bed-rest state (for example, whether the user P is in a bed-rest, bed-presence, bed-departure, edge sitting position, or the like) of the user, and also can acquire a sleeping state (sleeping, waking-up), as is described later.

The biosignal acquisition unit 400 in the present embodiment acquires a body vibration of a user by a pressure sensor, for example, and acquires breathing and heartbeat from the body vibration, but may acquire a biosignal by a load sensor from a change in the center of gravity position (body movement) of the user, may acquire a biosignal by a radar based on a displacement in a body surface or bedclothes, or may provide a microphone to acquire a biosignal based on sounds picked up by the microphone. The biosignal acquisition unit 400 only needs to acquire a biosignal of a user using any of the sensors.

In other words, the biosignal acquisition unit 400 may be connected with a device such as the detection device 12, or may receive a biosignal from an external device.

With the input unit 600, a measurement person inputs various conditions, and performs a manipulation input of a measurement start. The input unit 600 may be implemented by, for example, any of input units, such as a hardware key and a software key.

The output unit 700 is a functional unit that outputs a biological information value, such as a sleeping state, a heartbeat rate, and a respiratory rate, and makes a notification of an abnormality. The output unit 700 may be a display device, such as a display, or may be a notification device (sound output device) that makes a notification of a warning or the like. The output unit 700 may be an external storage device that stores data, or a transmission device or the like that transmits data through a communication path. The output unit 700 may be a communication device when a report is made to another device.

The input unit 600 and the output unit 700 may be implemented by other devices. For example, the input unit 600 and the output unit 700 may be implemented by using a terminal device (for example, a smartphone or a tablet that is used by the user) connected via the communicator 900. In this time, in the terminal device, the controller 100, which is described later, may be able to execute a program that implements the process.

The notification unit 800 makes a notification to a user or the like. For example, the notification unit 800 may be a speaker that outputs a sound, an LED serving as a light emitting device, or the like. The notification unit 800 may make a notification to another device (for example, a terminal device, such as a smartphone of a user, a nurse call, or the like).

The communicator 900 performs communication with another device. For example, the communicator 900 provides communication with a device in a short distance by a scheme, such as a wireless LAN (or a wired LAN), and Bluetooth (registered trademark). The communicator 900 may be a device that provides near field communication, such as NFC. The communicator 900 may provide communication by a scheme that allows mobile communication, such as 4G/LTE/5G/6G. The communicator 900 may be an interface (for example, a USB) or the like for performing communication with another device.

### [1.2.2 Software Configuration]

With reference to FIG. 3A, a configuration of software will be described. For example, the controller 100 implements the respective functions by executing programs and applications stored in the memory 200 (for example, the storage 210, the ROM 220, and the RAM 230).

A biological information value calculation unit 110 calculates biological information values (the respiratory rate, the heartbeat rate, the amount of activity, and the like) of the user P. In the present embodiment, the biological information value calculation unit 110 may extract a respiratory component and a heartbeat component from the body movement acquired by the biosignal acquisition unit 400, and obtain a respiratory rate and a heartbeat rate based on the breathing interval and the heartbeat interval. The biological information value calculation unit 110 may analyze (Fourier transform and the like) the periodicity of the body movement, and calculate a respiratory rate and a heartbeat rate from the peak frequency. The biological information value calculation unit 110 may calculate an amount of activity together. Specifically, the biological information value calculation unit 110 may detect a body vibration per sampling unit time from the biosignal acquisition unit 400, and calculate an amount of activity based on the number of times of the detected body vibrations. The biological information value calculation unit 110 may calculate an amount of activity from a change and a motion of a sleeping posture of a user.

Specifically, the biological information value calculation unit 110 continuously measures an output value from the sensor, for example, in a sampling period of 16 times per second (960 times per one minute). Predetermined measurement thresholds (an upper limit value and a lower limit value) are set to the measurement value of the sensor. The biological information value calculation unit 110 successively outputs the added-up amount of activity (0 to 960). The amount of activity is a numerical value amount (0 to 960) indicating the extent that the user P moves his/her body on the bed. The amount of activity relates to the frequency and the intensity of the body movement of the user P. The large amount of activity indicates that the user P frequently and largely moves his/her body on the bed.

A sleeping state determination unit 120 determines a sleeping state of a user. For example, the sleeping state determination unit 120 determines a sleeping state of a user based on the biosignal acquired by the biosignal acquisition unit 400. The sleeping state determination unit 120 may determine two states of “awake” and “sleep” as sleeping states. The sleeping state determination unit 120 may further determine the “sleep” state as “REM sleep” or “non-REM sleep”, and further make a determination of multiple levels (sleeping depths) as the “sleep” state.

The sleeping state determination unit 120 may determine the sleeping state and the awake state based on the magnitude of the amount of activity and the state of a time-series change in the amount of activity. For example, the sleeping state determination unit 120 does not need to determine the awake state if a temporal body movement is present. The sleeping state determination unit 120 may determine the awake state in a case where the body movement of the user P continues to some extent.

A user state acquisition unit 130 acquires a state of a user. A state of a user is a general state related to the user, and for example, a load sensor or the like provided to the bed 3 is used to acquire a state of whether the user is in a bed-departure or bed-presence state, for example. The user state acquisition unit 130 may further acquire, when the user is in a bed-presence state, a sleeping posture of the user and a sleeping position of the user. The user state acquisition unit 130 may acquire a state of the user based on the biosignal acquired in the biosignal acquisition unit 400, for example, as mentioned above, in addition to the load sensor or the like. The state of the user may include a state of whether the user is sleeping or awake based on the sleeping state of the user determined in the sleeping state determination unit 120. The user state acquisition unit 130 may acquire the bed-departure or the bed-presence of the user based on the amount of activity.

A user state detection unit 140 detects a state of a user from a parameter of a biological information value or the like. If the user state detection unit 140 has detected that the state of the user is in a predetermined state, the notification unit 800 may output an alert (notification).

The user state detection unit 140 in the present embodiment can detect whether the user has a symptom of cardiac failure aggravation from a value of a parameter to be obtained from biological information.

A disease information acquisition unit 150 acquires disease information that is information related to a disease of a user. The disease information acquisition unit 150 may be connected to an electronic medical record server, for example, and acquire disease information on the user. The disease information acquisition unit 150 may refer to information input by a medical doctor or the like, and acquire disease information. In the present embodiment, the disease information acquisition unit 150 acquires information indicating whether the user has a disease related to the cardiac failure as disease information.

The storage 210 stores biological information data 202, user state data 204, and a threshold table 206, and secures an area of a parameter buffer area 208.

The biological information data 202 stores, for example, a respiratory rate and a heartbeat rate, as information related to the biological information values calculated from the acquired biosignal (body movement) by the controller 100. In the present embodiment, as information related to the biological information values, a respiratory rate, a heartbeat rate, and a body movement are stored, but at least one of which may be stored. The biological information data 202 may further store other information (for example, a breathing event index based on the fluctuation or the like of the breathing amplitude or a periodic body movement index based on the periodicity of the body movement) as long as the information is a biological information value that can be calculated by the biological information value calculation unit 110. The biological information data 202 preferably store the biological information in a time-series manner for every predetermined time.

The user state data 204 stores a state of a user. The user state data 204 stores, as a state of a user acquired by the user state acquisition unit 130, whether the user is in a “bed-presence” or “bed-departure” state. The user state data 204 further stores the state of the user by including the sleeping state determined by the sleeping state determination unit 120. For example, if the user state acquisition unit 130 has determined that the user is in a “bed-presence” state, the sleeping state determined by the sleeping state determination unit 120 may be stored. Preferably, the user state data 204 store states of a user in a time-series manner for every predetermined time.

The threshold table 206 is a table in which a threshold to be compared with a parameter when the controller 100 detects whether a symptom of cardiac failure aggravation is present is stored. FIG. 3B illustrates one example of the threshold table 206.

In the present embodiment, parameters to be used for determining a symptom of cardiac failure aggravation in the user include a respiratory rate average value, a respiratory rate variation, a heartbeat rate average value, a heartbeat rate variation, and a heartbeat rate non-calculation rate. Biological information is used in order to calculate a value related to each parameter. In the present embodiment, biological information related to breathing and heartbeat is used, and the respiratory rate and the heartbeat rate serving as biological information values are used. The controller 100 uses data on the respiratory rate and data on the heartbeat rate that are output for every minute in the present embodiment, and as for average values of the respiratory rate and the heartbeat rate, average values during an evaluation section only need to be calculated, and the variations of the respiratory rate and the heartbeat rate only need to have a resolution to the extent that variations during a section of at least about 10 minutes can be calculated.

It can be considered that a section (calculation section, evaluation section) for calculating a value related to a parameter is set to, for example, a section 1 below as one section (for one day).
A time from the going-to-bed time to the wake-up time of a user. Examples thereof include the time during when the user is present on the bed 3, and the time during when biological information on the user is acquired.
A time during when the user is sleeping. Examples thereof include the time during when the user in the sleeping state, in the time during when biological information on the user is detected. The time when the user is awake during sleep may be included or may be excluded.
A section for every predetermined time, such as an interval of 10 minutes or 1 hour. In this case, a value is acquired by every arbitrary time unit, and at a stage when the measurement of values for one day (for example, 12 hours including night time, 24 hours) has ended, the section is redetermined as all night data, and a parameter may be recalculated again.
A predetermined time slot. Examples thereof include the section during the specified time such as the time from 23 o'clock to 6 o'clock and the time from 12 o'clock to 12 o'clock the next day.

The parameters to be calculated by the controller 100 are parameters as follows, for example.

### (1) Average Value

The average value is an average value of the biological information value of a user. In the present embodiment, average values of the calculated biological information values of the user for one day are used. As the biological information values, a respiratory rate and a heartbeat rate are used.

### (2) Variation

A variation is a variation of the biological information value of a user. As the biological information values, variations in the respiratory rate and the heartbeat rate are used.

Herein, as a calculation method of a variation, the variation is calculated by an expression indicated by the following expression (1).

$$\frac{1}{N} {\sum }_{n = 1}^{N} ( {P}_{n max} - {P}_{n min} ) \qquad ( 1 )$$

N: a number obtained by dividing the calculation section by the number of calculations of an epoch to be divided (an integer value, truncating the decimal point). For example, in the present embodiment, going-to-bed time of user/10 minutes (1 epoch=10 minutes) is set.
Pn max: the maximum value of a parameter of an n-th epoch.
Pn mix: the minimum value of a parameter of an n-th epoch.

Accordingly, the variation in the heartbeat rate or the respiratory rate for one day (for example, during the bedtime) is calculated. In addition to the above, a value of a variation calculated using the standard deviation or the standard error in the evaluation time may be used.

### (3) Heartbeat Rate Non-Calculation Rate

The heartbeat rate non-calculation rate is a ratio in which a heartbeat rate was not able to be calculated resulting from the evaluation of a low reliability of the heartbeat rate calculated using the measurement instrument in a predetermined period. The controller 100 determines whether the reliability of the calculated heartbeat rate is high (step S108). Whether the reliability of the calculated heartbeat rate is high may be determined by using the reliability evaluation methods described in JP2017-47211A (Application Date: Sep. 1, 2016, Title of Invention: BIOLOGICAL INFORMATION OUTPUT DEVICE, BIOLOGICAL INFORMATION OUTPUT METHOD, AND PROGRAM) and JP2019-97831A (Application Date: Nov. 30, 2017, Title of Invention: ABNORMALITY DETERMINATION DEVICE AND PROGRAM USED FOR THE SAME), for example. Herein, as one example of a calculation method of a heartbeat rate non-calculation rate, the heartbeat rate non-calculation rate is calculated by an expression indicated by the following expression (2).

$$\frac{{Act}_{0} ∩ H {R}_{null}}{{Act}_{0}} \qquad ( 2 )$$

Act₀: the time when the amount of activity becomes 0 within the evaluation time
HRₙᵤₗₗ: the time when a heartbeat rate was not calculated within the evaluation time

Herein, the evaluation time in the embodiment is, for example, a bed time from when a user enters in bed to when the user wakes up. The evaluation time may be the time from when a user goes to sleep to when the user is awake.

The controller 100 does not need to calculate all these parameters. However, the controller 100 preferably sets the respiratory rate average value as a necessary parameter. Combining a plurality of parameters can increase the detection accuracy of a symptom of cardiac failure aggravation.

The controller 100 may weight these parameters. For example, the controller 100 may detect a symptom of cardiac failure aggravation of a user by setting a coefficient to each parameter, and using a total value of the parameters multiplied by the coefficients.

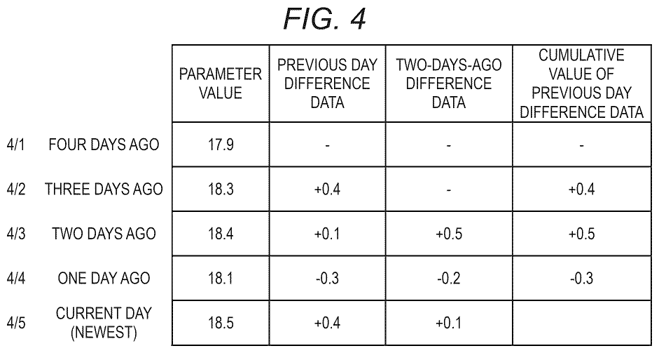

The parameter buffer area 208 is an area in which parameters are temporarily stored when aggravation of cardiac failure is determined based on the parameters. While daily data is stored in a time-series manner in the biological information data 202, parameters to be used in the processing are temporarily stored in the buffer area. For example, FIG. 4 is a diagram illustrating one example of the parameter buffer area 208. A parameter value of a given parameter on the current day, and parameter values one day ago, two days ago, three days ago, and four days ago are stored. The parameter buffer area 208 further stores a difference between the parameter value and that on the previous day, a two-days-ago difference, and a cumulative value of the previous day difference. Details of the parameter buffer will be described later.

### 1.3 Flow of Processing

Processing of detecting a symptom of cardiac failure aggravation of a user in the present embodiment will be described.

### [1.3.1 Overall Flow]

### (1) Main Process

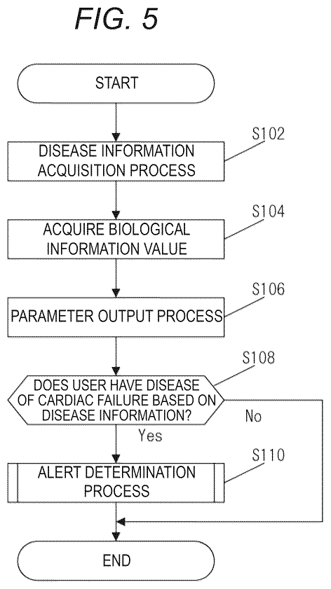

FIG. 5 is an operation flow illustrating the processing that is executed by the controller 100 when estimating a state of a user will be described. Firstly, the controller 100 executes a disease information acquisition process that acquires disease information on a user (S102). Herein, the controller 100 may preferably execute the present process with respect to a user having a disease of cardiac failure.

The controller 100 acquires a biological information value (S104). The controller 100 acquires a biological information value using the detection device 12, but may acquire it by using another wearable terminal device, for example.

Next, the controller 100 executes an output process of a parameter at predetermined timing (S106). The timing when the controller 100 calculates a value of the parameter is described above, but may output each parameter at predetermined timing, for example, for every 24 hours, or may output each parameter at predetermined timing including timing when the user wakes up (a parameter value calculated based on the going-to-bed time), for example.

If the controller 100 has determined that the user has a disease of cardiac failure based on the acquired disease information on the user (S108; Yes), the controller 100 then executes an alert determination process (S110).

### (2) Alert Determination Process, Cumulative Value Calculation Process

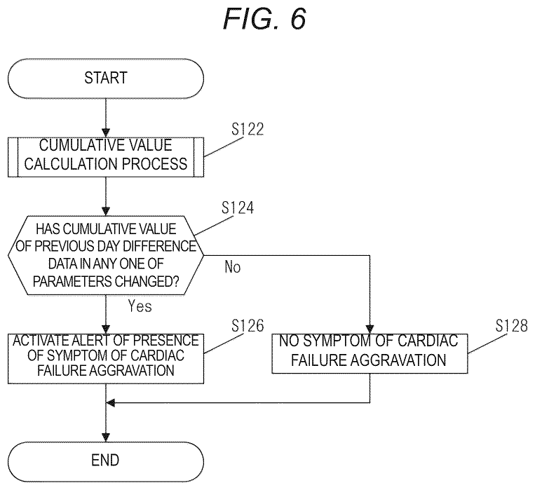

The alert determination process is executed, with reference to FIG. 6. The controller 100 executes the cumulative value calculation process, and calculates a cumulative value for each parameter (S122).

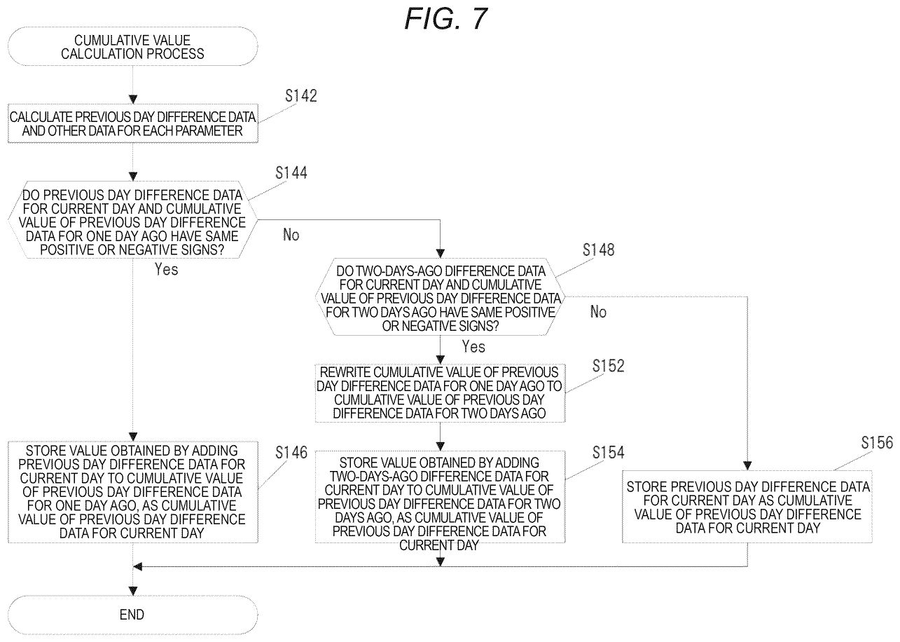

Herein, the cumulative value calculation process will be described with reference to FIG. 7. The calculation of a specific value will be described using the values stored in the buffer in FIG. 4 as examples.

As illustrated in the buffer in FIG. 4, a parameter value is firstly calculated and stored. For example, the controller 100 calculates a parameter value on the current day (the time of the calculation is used as a reference, day including the calculated day or day before 24 hours from the calculated time point, or day including a going-to-bed time from the wake-up on the calculated day to the entry-to-bed on the previous day), and stores the calculated parameter value for the current day. The controller 100 stores the parameter value already stored for the current day as that for one day ago. Similarly, values of the respective parameters in the previous four days are stored in the buffer.

For example, the controller 100 defines a day that is focused as a target of the calculation (the time of the calculation is used as a reference, day including the calculated day or day before 24 hours from the calculated time point, or day including a going-to-bed time from the wake-up on the calculated day to the entry-to-bed on the previous day), as the “current day”. The controller 100 calculates a value of a parameter related to the relevant “current day”, and stores the value as “current day data”. At that time, the controller 100 stores again the parameter value originally stored as the “current day data”, as “one-day-ago data”.

Herein, as mentioned above, parameter values are respectively calculated with respect to parameters of the respiratory rate average value, the respiratory rate variation, the heartbeat rate average value, the heartbeat rate variation, and the heartbeat rate non-calculation rate. FIG. 4 illustrates values of one parameter among the parameters. Hereinafter, values of one parameter will be described as an example.

The controller 100 calculates a previous day difference for each parameter (S142). For example, if the current day is 4/5, the controller 100 calculates a difference between a parameter value “18.5” which is current day data and a parameter value “18.1” which was calculated as one-day-ago data on 4/4, as previous day difference data “+0.4”.

The controller 100 may calculate a two-days-ago difference. For example, if the current day is 4/5, the controller 100 calculates a difference between the parameter value “18.5” for the current day and a parameter value “18.4” for 4/3 which is two days ago, as two-days-ago difference data “+0.1”.

As illustrated in FIG. 4, the controller 100 successively calculates previous day difference data and two-days-ago difference data, respectively. The controller 100 may calculate previous day difference data and two-days-ago difference data at any timing. For example, the controller 100 may calculate a value when being necessary in the following process, or may collectively calculate values at S142.

Referring back to FIG. 7, the controller 100 determines whether the previous day difference data calculated for the current day that is focused as an evaluation target has the same positive or negative sign as a cumulative value of previous day difference data (one day ago) calculated as a focus target for one day ago (S144). Herein, having the same positive or negative signs indicates that the positive or negative signs of the values are same, for example, and indicates signs of “+” and “+” or signs of “−” and “−”, for example. In other words, the controller 100 determines whether the parameter value monotonically increases (or monotonically decreases). In the present embodiment, having the same positive or negative signs may indicate a state other than a state of having the different positive and negative signs (for example, from positive to negative, from negative to positive). A case of zero is included in having the same positive or negative signs in which the signs of positive or negative are same.

If previous day difference data calculated for the current day has a positive or negative sign same as that of the cumulative value of the previous day difference data (one day ago) already calculated for one day ago, the controller 100 then stores a value obtained by adding the previous day difference data for the current day to the cumulative value of the previous day difference data (one day ago) already calculated for one day ago, as a cumulative value of the previous day difference data for the current day (S146).

If the previous day difference data for the current day and the cumulative value of the previous day difference data for one day ago have different positive and negative signs (S144; No), the controller 100 may add correction as follows in order to detect, at an earlier stage, a symptom of cardiac failure aggravation that repeatedly keeps getting better and then worse again, and is gradually aggravated. The controller 100 determines whether a difference between current day data serving as a focus target and two-days-ago data recorded two days ago and a cumulative value of the previous day difference data calculated two days ago have the identical positive or negative signs (S148). In other words, the controller 100 compares parameter values for two days ago and for the current day to grasp a tendency of the change, determines whether one-day-ago data is an outlier (irregular value) protruding from the tendency of the change, and adds a correction to the parameter value if the one-day-ago data is the outlier.

If the controller 100 has determined that the correction is necessary because the condition at S148 is satisfied (S148; Yes), the controller 100 rewrites the cumulative value of the previous day difference data for one day ago to the cumulative value of the difference data for two days ago (S152). The controller 100 then stores a value obtained by adding the two-days-ago difference for the current day to a cumulative value of the previous day difference for two days ago, as a cumulative value of the previous day difference data for the current day (S154).

If a positive or negative sign of the two-day-ago difference data for the current day is different from that of the cumulative value of the previous difference data for two days ago, the controller 100 stores the previous day difference data for the current day as a cumulative value of the previous day difference for the current day.

In other words, the controller 100 executes the cumulative value calculation process to appropriately calculate a cumulative value that is used for appropriately determining a trend of a parameter value (whether the parameter value increases or decreases).

In the above, the case has been described where the controller 100 performs the process at S148 if the controller 100 has determined that the previous day difference data for the current day and the cumulative value of the previous day difference data for one day ago have different positive and negative signs, and the parameter value does not monotonically increase (S144; No). However, in addition to the case, the controller 100 may reset the cumulative value, and newly store previous day difference data for the current day, as a cumulative value of the previous day difference data for the current day.

Referring back to FIG. 6, the controller 100 determines whether a cumulative value of the previous day difference data in any one of the parameters has changed (S124). Herein, specifically, the controller 100 determines whether the cumulative value is larger or less than the threshold stored in the threshold table 206.

If any one parameter among the plurality of calculated parameters matches the upper limit, the controller 100 determines that the user has a symptom of cardiac failure aggravation, and activates an alert (S124; Yes-S126). If the cumulative value of the previous day difference data is within the threshold in all the parameters, the controller 100 determines that the user has no symptom of cardiac failure aggravation (S124; No-S128).

The controller 100 may give notice to terminal devices (a smartphone, a computer, a tablet, and the like) of a person subjected to measurement himself/herself, a family, and a primary care doctor, when activating an alert at S126. The controller 100 may transmit the presence of a symptom of cardiac failure aggravation to an electronic medical record server, or output it to an associated digital cardiac failure notebook. If a symptom of cardiac failure aggravation is present, the controller 100 may give notice to a call center or the like, telephone a user for urging the user to go to the hospital or connect the user to a tele-nursing system.

### 1.4 Operation Example and Example

An operation example and an example of the cumulative value calculation process will be specifically described using the values of the parameter in FIG. 4.

(1) 4/2

When 4/2 is set as a reference day (current day), previous day difference data that is a difference between a parameter value “18.3” on 4/2 and a parameter value “17.9” on 4/1 as the previous day is “+0.4”. The cumulative value of the previous day difference data is “+0.4” at the time point of 4/2.

(2) 4/3

When 4/3 is set as a reference day (current day), previous day difference data that is a difference between a parameter value “18.4” on 4/3 and a parameter value “18.3” on 4/2 as the previous day is “+0.1”. Two-days-ago difference data that is a difference between the parameter value “18.4” on 4/3 and the parameter value “17.9” on 4/1 for two days ago is “+0.5”.

As for a cumulative value of the previous day difference data, the previous day difference data “+0.1” on 4/3 and the cumulative value “+0.4” of the previous day difference data on 4/2 for one day ago have the same “+” sign as a positive or negative sign. A cumulative value “+0.5” of the previous day difference data on 4/3 is obtained by adding “+0.1” to “+0.4”.

(3) 4/4

When 4/4 is set as a reference day (current day), previous day difference data that is a difference between a parameter value “18.1” on 4/4 and the parameter value “18.4” on 4/3 as the previous day is “−0.3”. Two-days-ago difference data that is a difference between the parameter value “18.1” on 4/4 and the parameter value “18.3” on 4/2 for two days ago is “−0.2”.

Herein, the previous day difference data “−0.3” on 4/4 and a cumulative value “+0.5” of the previous day difference data for one day ago have the different positive and negative signs. When two-days-ago difference data “−0.2” on 4/4 is compared with previous day difference data “+0.5” for two days ago, both have the different positive and negative signs. The controller 100 stores the previous day difference data “−0.3” on 4/4 as a cumulative value of the previous day difference data on 4/4.

(4) 4/5

When 4/5 is set as a reference day (current day), previous day difference data that is a difference between a parameter value “18.5” on 4/5 and the parameter value “18.1” on 4/4 as the previous day is “+0.4”. Two-days-ago difference data that is a difference between the parameter value “18.5” on 4/5 and the parameter value “18.4” on 4/3 for two days ago is “+0.1”.

Herein, the previous day difference data “+0.4” on 4/5 and a cumulative value “−0.3” of the previous day difference data for one day ago have the different positive and negative signs. When two-days-ago difference data “+0.1” on 4/5 is compared with the cumulative value “+0.5” of the previous day difference data for two days ago, the positive or negative signs are “+”, that is, same.

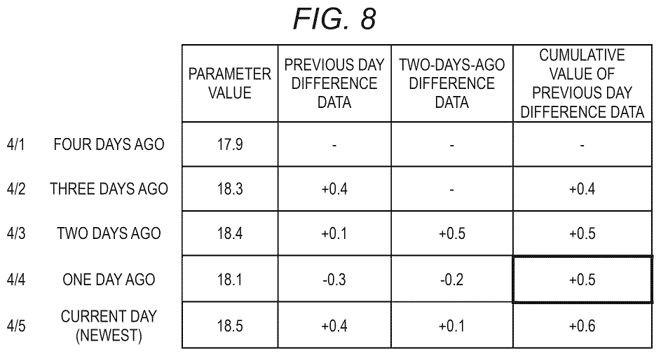

The controller 100 thus rewrites the cumulative value of the previous day difference data on 4/4 as one day ago to the cumulative value “+0.5” of the previous day difference data for two days ago. FIG. 8 is a diagram illustrating a state of the rewritten buffer. As illustrated with the thick frame in FIG. 8, the cumulative value of the previous day difference data on 4/4 is rewritten to “+0.5”.

The controller 100 then stores a value obtained by adding two-days-ago difference data “0.1” to the cumulative value of the previous day difference data on 4/3 as two days ago, as a cumulative value of the previous day difference data on 4/5.

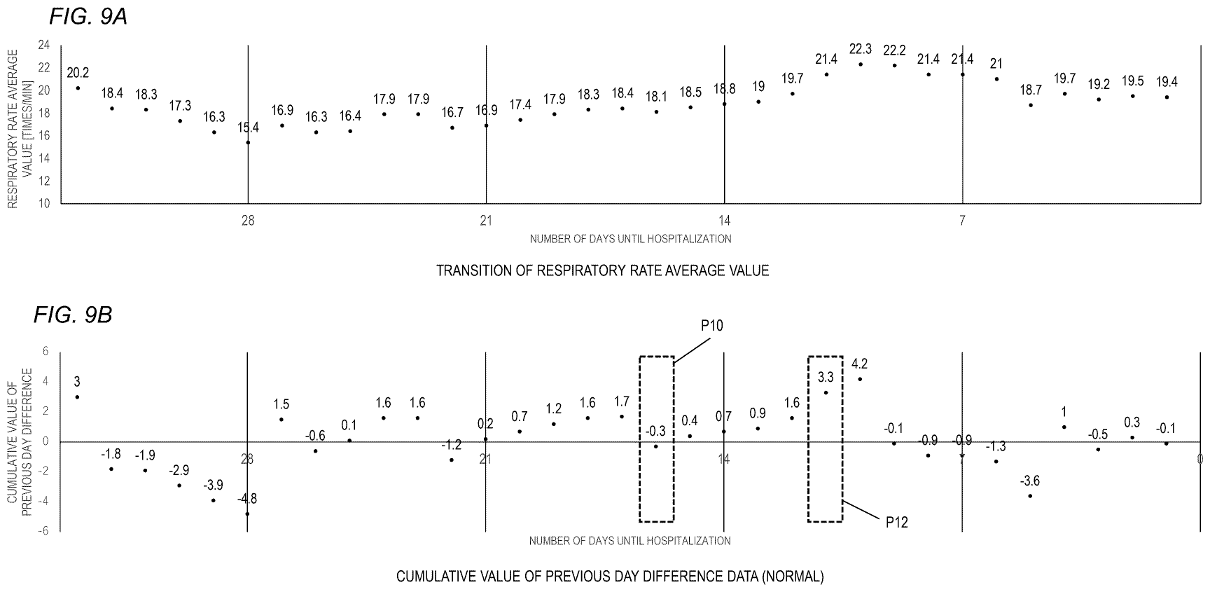

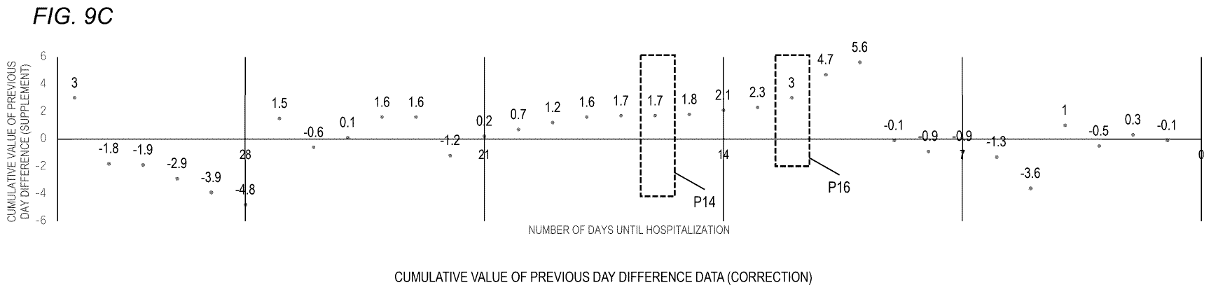

FIG. 9 illustrates a plotted transition of a parameter. The average value of the respiratory rate is used as the parameter in FIG. 9. Parameter values of the respiratory rate average value are respectively plotted in FIG. 9A. FIG. 9B is a graph in which cumulative values of the previous day difference data are plotted, and FIG. 9C is a diagram to which the processing in the embodiment is applied. FIG. 9A illustrates the measurement data of a cardiac failure patient, and an x-axis indicates the number of days until the patient whose data has been measured is hospitalized because of the cardiac failure aggravation (0 at the right end of the graph indicates a hospitalized day).

For example, as illustrated by a dashed line P10 in FIG. 9B, the cumulative value of the previous day difference data indicates “−0.3” once, a rising trend of the parameter value is interrupted. In contrast, after the correction in FIG. 9C, as illustrated by the dashed line, the cumulative value of the previous day difference data is corrected to “1.7”. This results in a state where the rising trend of the parameter value continuously appears. For example, a cumulative value “3” of the previous day difference data is obtained in a dashed line P16 that is 12 days before the hospitalization, so that the cardiac failure aggravation in the user can be detected. In FIG. 9B, as for the cumulative value of the previous day difference data at the corresponding day, a cumulative value “3.3” of the previous day difference data is obtained in a dashed line P12 that is 11 days before the hospitalization, and the cardiac failure aggravation is detected late by 1 day in comparison with that in FIG. 9C. Finding cardiac failure aggravation at an early stage has an effect on the prognosis, and the earlier detection even by one day is effective, so that using a correction method can detect a change in the parameter that shifts at an earlier stage and in a long term, regardless that using no correction method has a certain effect.

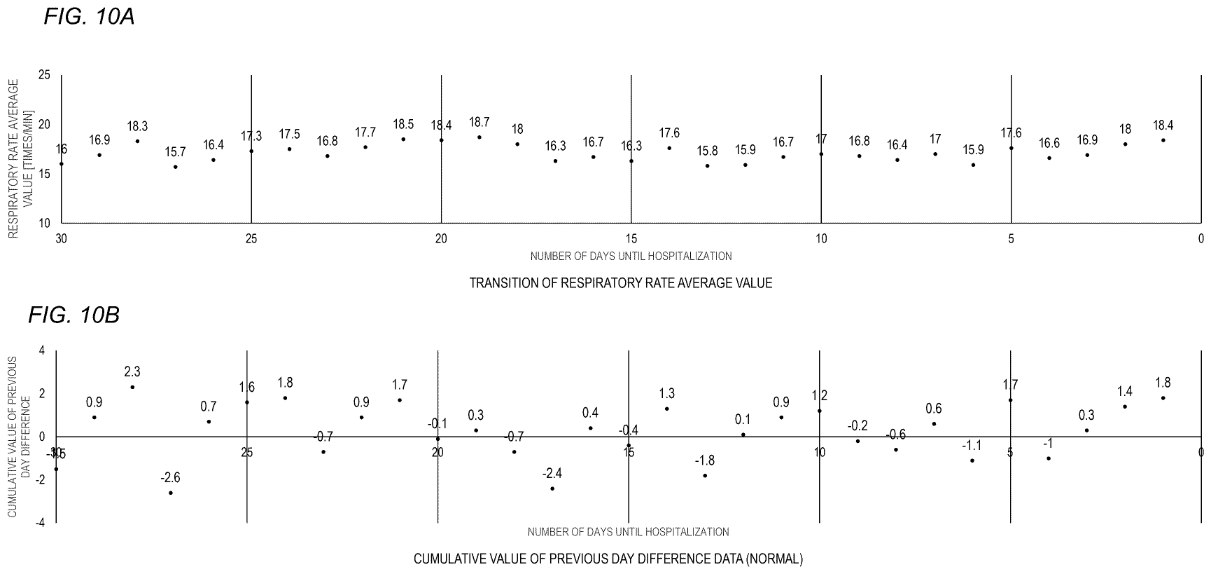

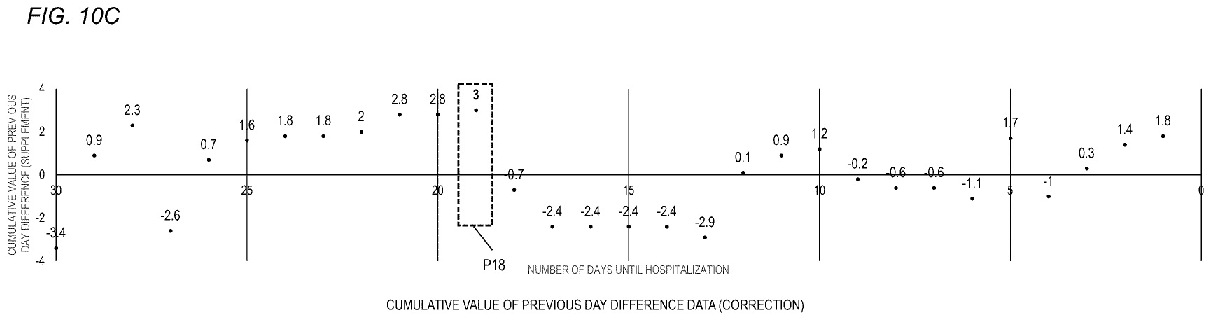

An operation example in FIG. 10 is a graph based on an average value of the respiratory rate as a parameter, similarly to FIG. 9. In FIG. 10A, the average value of the respiratory rate is used as a parameter, and parameter values of the respiratory rate average value are respectively plotted. FIG. 10B is a graph in which cumulative values of the previous day difference are plotted without any change, and FIG. 10C is a diagram to which the processing in the embodiment is applied. In the example in FIG. 10, while cardiac failure aggravation cannot be detected in FIG. 10B, cardiac failure aggravation can be detected in FIG. 10C.

### 1.5 Effects and the Like

With the present embodiment in this manner, a symptom of cardiac failure aggravation of a user can be appropriately detected by using a parameter. When a symptom of cardiac failure aggravation is detected, the controller 100 activates an alert to allow a medical doctor and a staff, for example, to notice a state of the user earlier.

A symptom of cardiac failure aggravation in the user can be detected without delay to result in early treatment, which can lead to the improvement in the prognosis and the prevention of decreased QOL, in comparison with a case of the emergency hospitalization after the development of symptoms.

As for the system in the embodiment, the verification was performed for 18 cases of patients who were hospitalized due to the cardiac failure aggravation and received the urgent medical examination (intravenous diuretic injection), and 4 cases who were hospitalized due to non-cardiac failure. Based on the patients, a threshold of the parameter was adjusted so as to obtain the sensitivity of 100% for 18 cases, and the specificity of 100% for 4 cases.

As a result, extremely excellent effects were able be obtained, that is, the false report rate of 4.1% as a result that the activation of an alert in the detection system according to the present embodiment was applied to seven subjects who have been never hospitalized due to cardiac failure, in 3987 days in total.

A possibility that a symptom of cardiac failure aggravation cannot be captured from data resulting from daily measurement of the blood pressure and the body weight of a patient with cardiac failure is suggested. It is considered that direct biological information (vital information) serves as an effective indicator, specially a respiratory rate for a patient with cardiac failure who has a disease in his/her heart, in order to detect a physical condition of the user. Patients with cardiac failure have different allowable ranges of the pain of symptoms in the subjective impressions, and tolerate the symptoms, which leads to the hospitalization in many cases. In order to capture a symptom of cardiac failure aggravation, monitoring with an objective indicator to capture the aggravation symptom is effective.

The aggravation of cardiac failure includes a case of sudden worse change, and a case where the aggravation is progressed over several weeks in the long term, so that specialized parameters and thresholds for detecting a symptom of cardiac failure aggravation are necessary, unlike a simple physical condition change.

When a measurement target (detection object) is a user (patient) having a disease of cardiac failure, the detection apparatus and the detection system in the embodiment are used. The detection apparatus in the embodiment can detect the presence of a symptom of cardiac failure aggravation when the transitions of the average value of the respiratory rate, the average value of the heartbeat rate, the variation of the respiratory rate, the variation of the heartbeat rate, and the non-calculation rate of the heartbeat rate have exceeded thresholds.

### 2. Second Embodiment

Subsequently, a second embodiment will be described. The case where a determination is made based on the cumulative value in order to determine a so-called outlier has been described in the above-described embodiment. The present embodiment is an embodiment in which an entire trend of the parameter value is obtained by a different method.

The second embodiment is the same as the first embodiment in the hardware configuration and the configuration of main software, and only different points from the first embodiment will be described.

### (1) Method of Obtaining Correction Using Inclination of Regression Straight Line

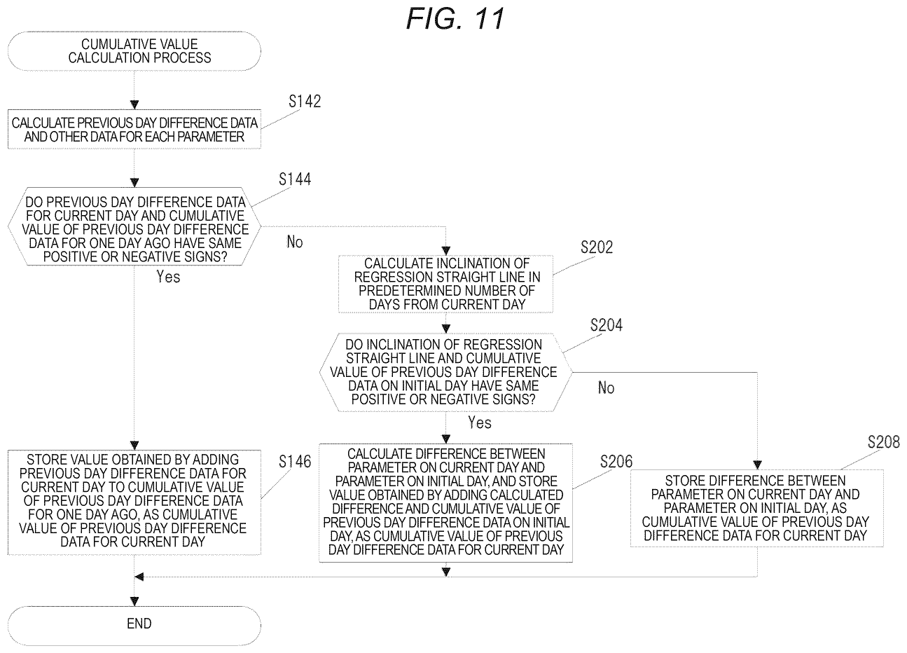

A description is made with reference to FIG. 11. The controller 100 calculates a previous day difference data and the like for each parameter. Herein, the controller 100 may calculate a previous day difference data, a two-days-ago difference data, and the like, and calculate a cumulative value of the previous day difference data until the previous day. The description of these calculations has been described in the first embodiment, and is thus omitted. FIG. 11 is a flow based on FIG. 7, and the same process is denoted by the same reference numeral, and the description thereof is omitted.

Herein, the controller 100 determines whether previous day difference data for the current day and the cumulative value of the previous day difference data for one day ago have the same positive or negative signs (S144). If the previous day difference data for the current day and the cumulative value of the previous day difference data for one day ago have the same positive or negative signs, the controller 100 stores a value obtained by adding the previous day difference data for the current day to the cumulative value of the previous day difference data for one day ago, as a cumulative value of the previous day difference data for the current day (S144; Yes-S146). If the previous day difference data for the current day and the cumulative value of the previous day difference data for one day ago have different positive and negative signs, the controller 100 calculates an inclination of the regression straight line in a section went back by the predetermined number of days from the current day (for example, three days, five days, or the like including the current day) (S202).

The controller 100 determines, when calculating a regression straight line, whether a cumulative value of the previous day difference data on a day that is the most distant from the current day, among the predetermined number of days that is the section in which the regression straight line is calculated (For example, two days ago in the case of three days including the current day, the oldest day in comparison with the current day. The first day in the predetermined number of days that is the section in which the regression straight line is calculated is hereinafter called “initial day”.), and the inclination of the regression straight line have the same positive or negative signs (S204).

If the positive or negative signs determined at S204 are same (S204; Yes), the controller 100 assumes an approximate increase or decrease from the initial day to the current day in the predetermined number of days used for the calculation of the regression straight line, and calculates a difference between the parameter for the current day and the parameter for the initial day. The controller 100 then stores a cumulative value of the previous day difference data obtained by adding the calculated difference to the cumulative value of the previous day difference data for the initial day, as the cumulative value of the previous day difference data for the current day (S206).

If the positive or negative signs determined at S204 are not same (S204; No), the controller 100 stores the difference between the parameter for the current day and the parameter for the initial day, as a cumulative value of the previous day difference data for the current day (S208).

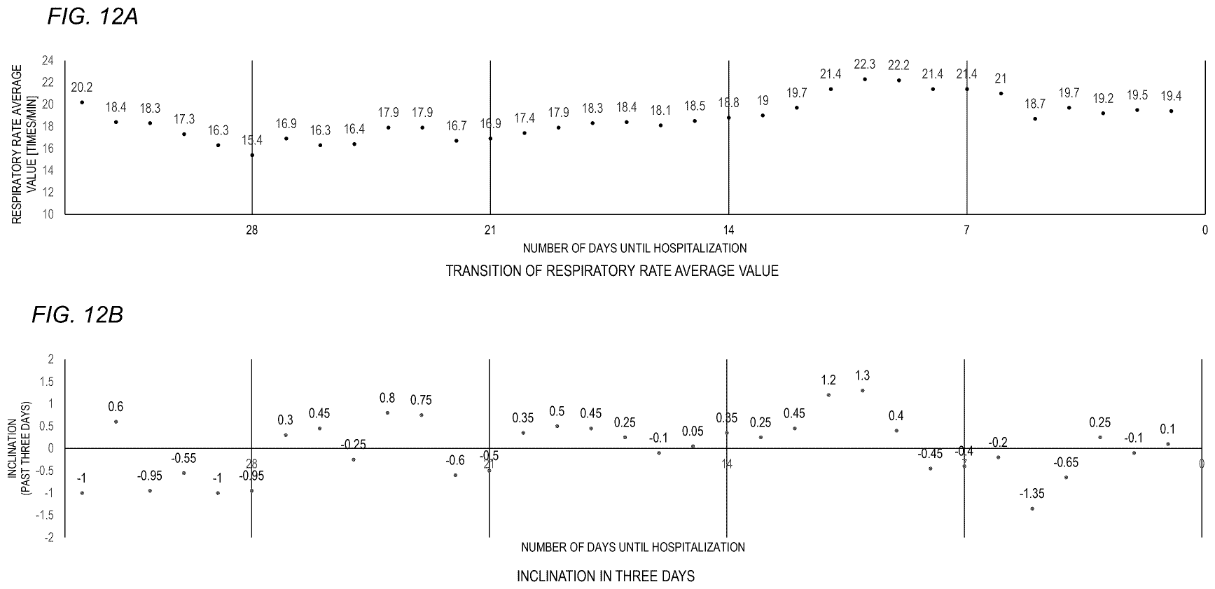

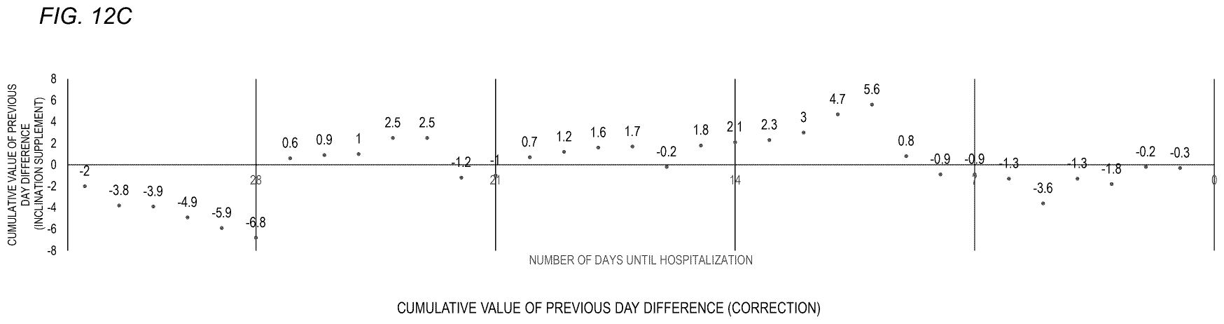

For example, FIG. 12A is a graph illustrating a transition of average values of the respiratory rate. FIG. 12B is a graph illustrating an inclination in three days.

(2) Method of Obtaining Cumulative Value of Previous Day Difference Data after Correction Using Moving Average

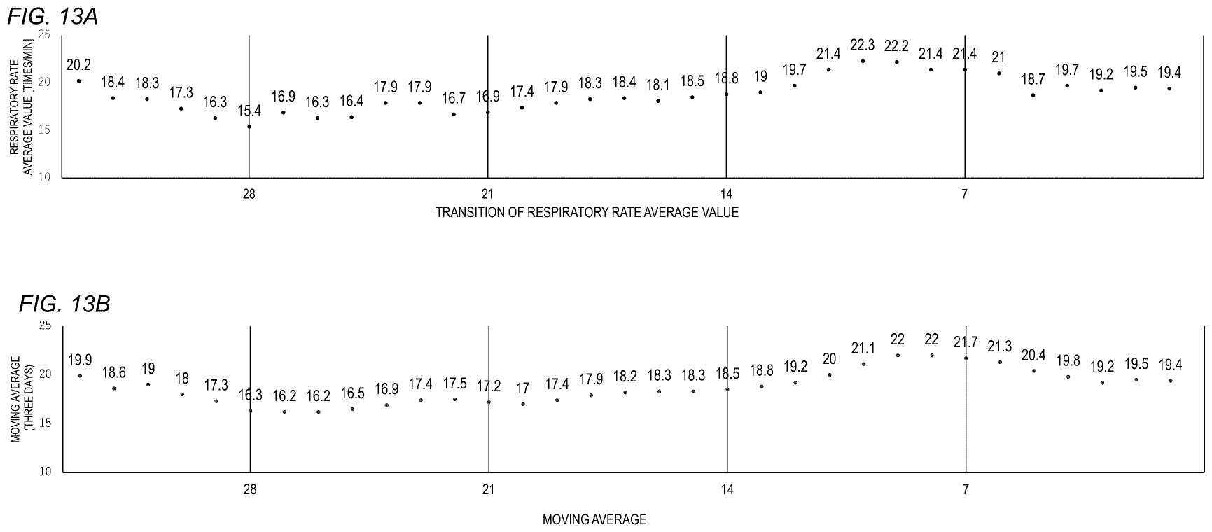

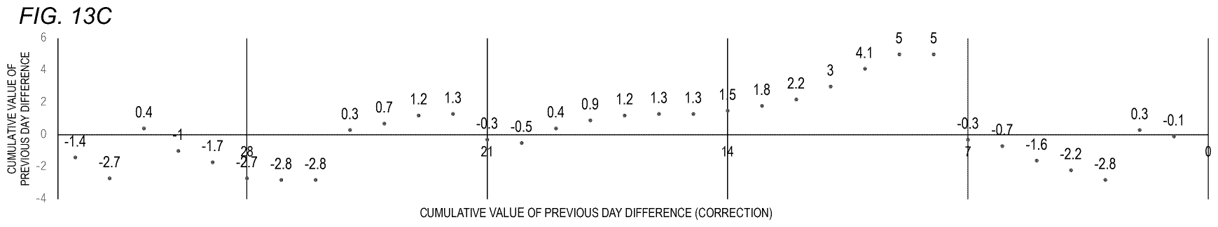

The controller 100 calculates an average value in a predetermined number of days including the current day (for example, in a case of past three days, the current day, the previous day, and two days ago) (hereinafter, referred to as a moving average value) for each parameter. FIG. 13A is a graph in which the average value of the respiratory rate is plotted for each day, and FIG. 13B is a graph in which a moving average in three days is calculated and plotted.

The controller 100 further calculates a previous day difference data using the moving average value. The controller 100 calculates, with respect to a previous day difference data calculated from the moving average value, a cumulative value of the previous day difference data. FIG. 13C is a graph in which the cumulative value of the previous day difference data is plotted. In a case where the moving average value is used, a correction does not need to be added when a cumulative value of the previous day difference data is calculated. When the cumulative value of the previous day difference data calculated in each parameter has exceeded a threshold, the presence of a symptom of cardiac failure aggravation can be detected.

(3) Method of Obtaining Difference from Arbitrary Distant Day

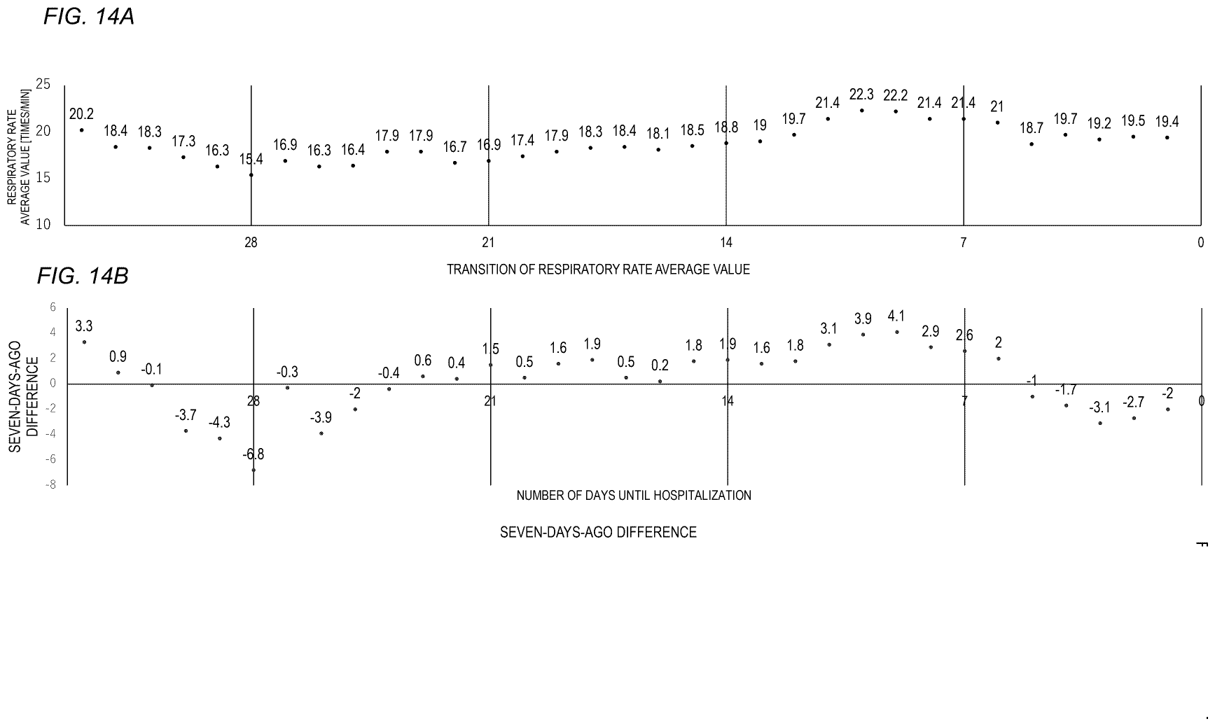

The controller 100 calculates a difference between the current day and a predetermined number of days ago (for example, seven days ago) for each parameter. This calculates a changing amount in comparison with a predetermined number of days ago. For example, FIG. 14B is a graph in which the average value of the respiratory rate is plotted for each day, and FIG. 14B is a graph illustrating a difference from seven days ago. Seven days ago is set as one example of a predetermined number of days in FIG. 14B, and it is desired to set the predetermined number of days as days over the past five days, because the changing amount in one to two days is small in a case where the parameter continuously increases or decreases. In this time, when the difference from the predetermined number of days ago calculated in each parameter has exceeded a threshold, the presence of a symptom of cardiac failure aggravation can be detected.

### 3. Third Embodiment

A third embodiment will be described. The example in which the processes are respectively executed in parameters, and if even one of the parameters exceeds the value, the presence of a symptom of cardiac failure aggravation is detected has been described in the first embodiment.

In the present embodiment, an embodiment in which the controller 100 weights a calculated parameter, and makes a determination of cardiac failure aggravation will be described.

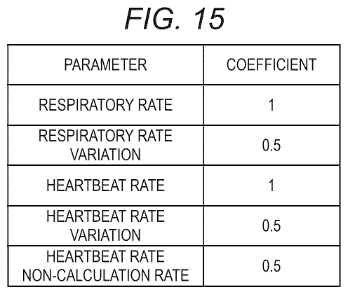

For example, FIG. 15 is a diagram illustrating one example of a table in which a weighting coefficient for each parameter is stored. For example, a parameter having exceeded a threshold is set as 1 and multiplied by a weight coefficient, and the presence of a symptom of cardiac failure aggravation may be determined if the sum of all the parameters has exceeded 1. For example, in the example in FIG. 15, the respiratory rate and the heartbeat rate each have a coefficient of 1, so that the controller 100 detects the presence of a symptom of cardiac failure aggravation in the user at a time point when a cumulative value of the previous day difference data of at least the respiratory rate and/or the heartbeat rate has exceeded a threshold.

The respiratory rate variation, the heartbeat rate variation, and the heartbeat rate non-calculation rate each have a coefficient of 0.5, so that the controller 100 does not detect the presence of symptom of cardiac failure aggravation when the cumulative value of the previous day difference data in one of the parameters has exceeded a threshold, and the cumulative value of the previous day difference data in the other parameters needs to exceed a threshold.

In the determination as to whether the cumulative value of the previous day difference data in the parameter exceeds a threshold, if the sum in an arbitrary period (for example, for three days) has exceeded 1, the controller 100 may detect the presence of a symptom of cardiac failure aggravation.

The controller 100 may perform weighting by another method. For example, if the cumulative value of the previous day difference data has exceeded (become less than) a determination threshold, the controller 100 obtains coefficient×cumulative value/determination threshold, as a calculation value for each parameter. If the sum of the calculation values has exceeded 1, the controller 100 then detects the presence of a symptom of cardiac failure aggravation in the user. In this time, the controller 100 may evaluate the higher risk of cardiac failure aggravation as the total value is larger.

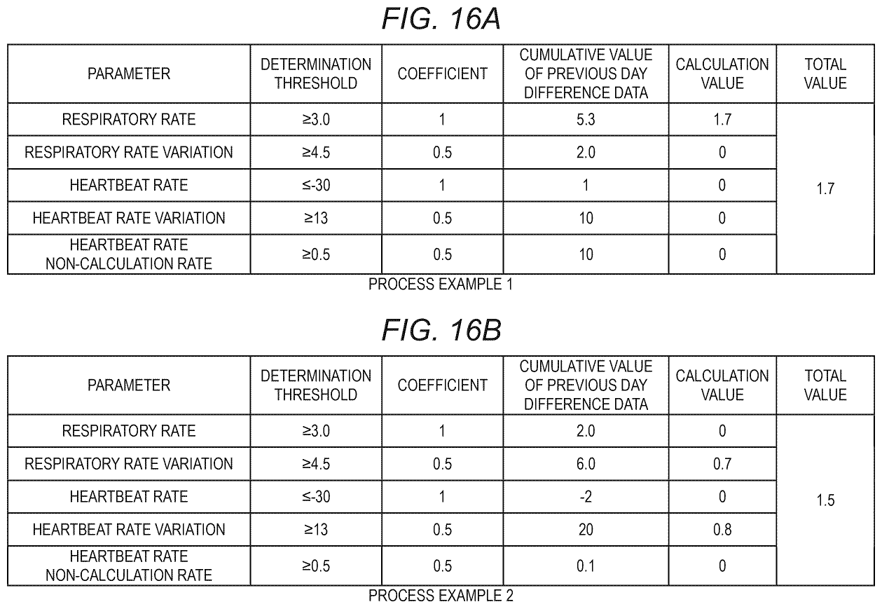

FIGS. 16A and 16B illustrate process examples described using specific numerical values. For example, in FIG. 16A, the cumulative value of the previous day difference data exceeds a determination threshold only in the respiratory rate. The controller 100 obtains 1×5.3/3.0=1.7 as a calculation value. In FIG. 16A, 1.7 serves as a total value without any change.

In FIG. 16B, the cumulative value of the previous day difference data exceeds a determination threshold in the respiratory rate variation and the heartbeat rate variation. The controller 100 calculates “0.5×6/4.5=0.7” and “0.5×20/13=0.8” as calculation values. The controller 100 then calculates the sum of the calculation values of 0.7+0.8=1.5, as a total value.

In this manner, with the present embodiment, the controller 100 can detect a symptom of cardiac failure aggravation in a user by combining a plurality of the calculated biological information values.

### 4. Modification Example

The present disclosure is not limited to each of the above-described embodiments, and various modifications can be made thereto.

That is, embodiments that can be implemented by combining technical means appropriately modified within the scope that does not depart from the gist of the present disclosure are also included in the technical scope.

The above-described embodiments are divided for convenience of the description. However, the embodiments can be combined and implemented within the realm of possibility. The applicants has an intention to acquire the right for any of the techniques disclosed in the present disclosure in the forms of an amendment, a divisional application, and the like.

Moreover, the program that operates in each device in the respective embodiments is a program that controls a CPU or the like (a program that causes a computer to function) so as to implement the functions in the abovementioned embodiments. Further, the information that is handled by these devices is temporarily stored in a temporary storage (for example, RAM) at the time of processing, and thereafter stored in various ROM and HDD storage devices, and is read and corrected/written by the CPU as necessary.

Herein, as a recording medium that stores the program, any of a semiconductor medium (for example, ROM and a nonvolatile memory card), an optical recording medium, a magneto-optical recording medium (for example, a digital versatile disc (DVD), a compact disc (CD), and a Blu-ray (registered trademark) disc (BD)), a magnetic recording medium (for example, a magnetic tape and a flexible disc), and the like may be used.

Further, in a case of distribution to the market, the program can be stored and distributed in a portable recording medium, or transferred to a server computer connected via a network such as the Internet. In this case, a storage device of the server device, of course, falls within the present disclosure.

The above-described data is not stored in the device, but may be stored in an external device, and called, as appropriate. For example, data may be stored in a network attached storage (NAS), or stored on a cloud.

The scope of the present disclosure is not limited to the configurations that are explicitly described in the present disclosure, and includes combinations of the techniques disclosed in the present disclosure. In the present disclosure, the configurations, for which the applicant attempts to acquire a patent, are described in the claims. However, it is not intended that the configuration not described in the claims is excluded from the technical scope.

In the above-described specification, each of the expressions such as “in the case where . . . ” and “when . . . ” only represents one example, and thus the present disclosure is not limited to the contents described above.

Configurations not described with these expressions are also disclosed within the scope that is apparent for those skilled in the art, and the applicant has an intention to acquire the right for such configurations.

Orders of the processing and the data flow described in the present specification are not limited to the described orders. For example, the configuration in which the processing is partially omitted and the configuration in which the order of the processing is changed are also disclosed, and the applicant has an intention to acquire the right therefor.

In the description, the functions described in the embodiments are executed in the respective devices, but may be implemented in one device, and an external server may further be used.

The respective functional blocks or various features of the devices used in the above-described embodiments can be implemented or executed by an electric circuit, for example, an integrated circuit or a plurality of integrated circuits. Electrical circuits designed to execute the functions described in the present specification may include general purpose processors, digital signal processors (DSPs), application specific integrated circuits (ASICs), field programmable gate arrays (FPGAs), or other programmable logic devices, discrete gate or transistor logic, discrete hardware components, or combinations thereof. The general purpose processor may be a microprocessor or a processor, a controller, a microcontroller, or a state machine of the related art. The above-described electric circuit may be implemented by a digital circuit or may be implemented by an analog circuit. In a case where a technology of an integrated circuit that replaces the current integrated circuit appears due to the progress of the semiconductor technology, one or more aspects in the present disclosure can also use a new integrated circuit based on the technology.

The processor 14 outputs biological information based on a result output from the detection device 12 in the present embodiment, however, the detection device 12 may calculate the whole. An application is installed and implemented not only in a terminal device (for example, a smartphone, a tablet, and a computer), but also processing may be performed at a server side and a process result may be returned to the terminal device, for example.

For example, the detection device 12 may upload biological information to the server, thereby implementing the above-described processing at the server side. The detection device 12 may be implemented by a device such as a smartphone in which an acceleration sensor and a vibration sensor are incorporated, for example.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A detection apparatus comprising:
a biosignal acquisition unit configured to acquire a biosignal of a user; and
a controller, wherein
the controller is configured to
calculate one or more parameter values related to breathing of the user based on the biosignal acquired from the biosignal acquisition unit, and
detect a presence of a symptom of aggravation of cardiac failure in the user when a calculated parameter value in which a cumulative value of previous day difference data has exceeded a predetermined threshold is present among the calculated parameter values.

2. The detection apparatus according to claim 1, wherein
the controller is configured to
calculate a difference between current day data serving as the parameter value on a reference day and one-day-ago data serving as a parameter value on a previous day of the reference day, as previous day difference data on the reference day, and
calculate a cumulative value of the previous day difference data by cumulatively adding the previous day difference data until the reference day.

3. The detection apparatus according to claim 2, wherein the controller is configured to calculate, when a previous day difference data for the current day and a cumulative value of the previous day difference data for one day ago have same positive or negative signs, the cumulative value of the previous day difference data.

4. The detection apparatus according to claim 2, wherein
the controller is configured to
compare, when the previous day difference data for the current day and the cumulative value of the previous day difference data for one day ago have different positive and negative signs, signs of a difference between the parameter values for the current day and two days ago from the current day and a cumulative value of the previous day difference data for two days ago,
replace, when the difference between the parameter values for the current day and two days ago from the current day and the cumulative value of the previous day difference data for two days ago have the same signs, the cumulative value of the previous day difference data for one day ago with the cumulative value of the previous day difference data for two days ago.

5. The detection apparatus according to claim 1, wherein the controller is configured to calculate, as the parameter values related to the breathing of the user, an average value of a respiratory rate and/or a variation of the respiratory rate.

6. The detection apparatus according to claim 1, wherein the controller is configured to further calculate a parameter value related to heartbeat of the user.

7. The detection apparatus according to claim 6, wherein the controller is configured to calculate, as the parameter value related to the heartbeat of the user, at least any one of an average value of a heartbeat rate, a variation of the heartbeat rate, and a heartbeat rate non-calculation rate.
